(19) United States
(12) Patent Application Publication
(10) Pub. No.: US 2011/0044900 A1
(43) Pub. Date: Feb. 24, 2011

(54) METHOD FOR TREATING AND/OR DIAGNOSING TUMOR BY GOLD PARTICLES COATED WITH A POLYMER

(75) Inventors: Yeu-Kuang Hwu, Taipei (TW); Chang-Hai Wang, Taipei (TW); Chi-Jen Liu, Taipei (TW); Cheng-Liang Wang, Taipei (TW); Chi-Hsiung Chen, Taipei (TW); Chung-Shi Yang, Miaoli (TW); Hong-Ming Lin, Taipei (TW); Jung-Ho Je, Pohang (KR); Giorgio Margartondo, Renens (CH)

Correspondence Address: PAI PATENT & TRADEMARK LAW FIRM, 1001 FOURTH AVENUE, SUITE 3200, SEATTLE, WA, 98154, US

(73) Assignee: Yeu-Kuang Hwu, Taipei (TW)

(21) Appl. No.: 12/545,824

(22) Filed: Aug. 22, 2009

(51) Int. Cl. A61K 49/00; A61K 33/24; A61P 35/00; A61K 9/16

(52) U.S. Cl. 424/9.1; 424/649; 424/497; 977/810

(57) ABSTRACT

A method for treating and/or diagnosing a tumor is provided. The method includes administrating an effective amount of gold particles to a subject in need thereof, and observing the distribution of the gold particles in the subject, wherein the gold particles are coated with a polymer, and the gold particle has a size of about 6.1±1.9 nm.

### BACKGROUND OF THE INVENTION

1. Field of the Invention

The present invention relates to the treatment of cancer, and in particular relates to a method for treating and/or diagnosing a tumor by gold particles coated with a polymer.

2. Description of the Related Art

Colloidal nanoparticles are utilized as active elements in biosensor, bio-imaging and tumor treatment biomedical applications.

The use of nanoparticles for cancer therapy as drug carriers or radiotherapy enhancers strictly depends on the selective accumulation of the nanoparticles in tumors. Accumulation of nanoparticles is the result of the enhanced permeation and retention (EPR) effect due to the vascular leakage and abnormal vessel architecture of cancerous areas. Thus, long-term retention of nanoparticles in the tumor is important, since the decreases the nanoparticles concentration in normal areas reduces the risk of their damage by cancer therapy.

When utilizing gold nanoparticles in cancer therapy, the gold nanoparticles quickly dissipate in cancerous areas, because of the phagocytosis of the macropharge or immune cells. Thus, required absolute concentrations are difficult to achieve by utilizing a simple tail vein injection. To increase the accumulation of the nanoparticles, a PEG modification is utilized. The surface modification of the PEG can increase accumulation time to several hours.

However, in previous studies, the PEGlyated gold nanoparticle is prepared by PEG-thiol and/or their derivatives to modify the surfaces of pre-synthesized citrated reduced gold nanoparticle. Thus, the conventional PEGlyated gold nanoparticles contain various reducing agents, surfactant, or other chemical compounds to hinder reduction of the gold nanoparticles. The reducing agents and surfactant however, may lead to the cytotoxicity of healthy tissues. For example, the chemical compounds may damage cell DNA or cause cancer. Further, the chemical compounds also may lead to environmental pollution.

To circumvent the previously mentioned problems, a novel particle and method for treating cancer is required.

### BRIEF SUMMARY OF THE INVENTION

The invention provides a method for treating and/or diagnosing a tumor, comprising administrating an effective amount of gold particles to a subject in need thereof, and observing the distribution of the gold particles in the subject, wherein the gold particles are coated with a polymer, and the gold particle has a size of about 6.1±1.9 nm.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1a shows a TEM micrograph of the gold particles of the invention;

FIG. 1b shows the PEG-gold particles of the invention with a 6.1±1.9 nm in diameter and reasonable size distribution.

FIG. 1c is X-ray diffraction (XRD) measurements of the crystal nature of PEG-gold particles of the invention;

FIG. 1d is a fourier transform infrared (FTIR) spectrum of a PEG-gold particle of the invention;

FIGS. 2a-2b show that large amounts of PEG-gold particles are internalized in the cytoplasm;

FIG. 2c is a graph plotting cellular uptake against particle concentration;

FIG. 2d shows that the particle of the invention is not toxic;

FIGS. 3a-3e are graphs plotting X-ray dosage against cell colony number;

FIGS. 4a-4c show the time-dependent distribution profiles of PEG-gold colloidal at different administrated doses;

FIG. 5 shows a visual examination of the tumors;

FIG. 6a-6f shows a time sequence of microradiographs extracted from real-time video sequence taken during and after the injection of PEG-gold particles;

FIGS. 7a-7f show TEM micrographs of various mice organs and tumors;

FIGS. 8a-8f show microscopy images of tumor, spleen, liver and lung, kidney, and muscle after H-E staining, and

FIG. 9 shows that PEG-gold particle enhances the suppression of the tumor growth.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides a gold particle. The gold particle comprises a polymer coating on a surface of the particle, wherein the polymer coating does not comprise thiol groups.

The surface of the gold particle of the invention is coated with a polymer. The polymer includes PEG, PEI, PVP, IPA, or a combination thereof. In one embodiment, the particle of the invention is a gold PEGylated particle. In another embodiment, the particle of the invention is a gold PVPylated particle. The size of the particle of the invention is about 6.1±1.9 nm, and the particles of the invention have superior dispersion, shape, and size.

Note that the particle of the invention is a thiol-free particle. The gold particles of the invention may be prepared by using a high energy and high flux radiation process. For example, a precursor solution containing PEG, PEI, PVP, or IPV is irradiated with an ionizing radiation beam with high energy and high flux to convert the precursor to the particle coated with PEG, PEI, PVP, or IPV. The concentration ratio of the PEG, PEI, PVP, or IPA to the precursor may be about 0.0001:0.12. The molecular weight of the PEG may be between 1000 and 250000. Since no reducing agent or stabilizer is required in the precursor solution, the particle colloidal is clean and non-toxic, and the thiol-free particle is more suitable for biomedical applications.

Further, the gold particle of the invention has superior stability and uniform size (less than 100 nm) so that the gold particle of the invention can be significantly aggregated in the cancerous cell or tumor by EPR (enhanced permeation and retention) effect.

The thiol-free particle is used to treat and/or diagnosis tumors. The invention further provides a method for treating and/or diagnosing a tumor. The method comprises: administrating an effective amount of the gold particles of the invention to a subject in need thereof, and irradiating the tumor with a radiation. The radiation is applied to provide cancer radiotherapy or a radiology diagnosis.

The term “tumor” refers to an abnormal benign or malignant mass of tissue that is not inflammatory and possesses no physiological function. Generally, the tumor occurs in the organ selected from the group consisting of breast, lung, brain, liver, skin, kidney, GI organ, prostate, bladder, gynecological organ and any other hollow organ. The tumor comprises breast cancer, a lung cancer, a brain cancer, a liver cancer, a skin cancer, a kidney cancer, a GI cancer (gastric, colon and rectal carcinoma), a prostate cancer, a bladder cancer, or a gynecologic cancer (cervical, ovarian, uterine, vaginal, and vulvar carcinoma).

The “subject” of the invention refers to human or non-human mammal, e.g. a dog, a cat, a mouse, a rat, a cow, a sheep, a pig, a goat, or a primate, and expressly includes laboratory mammals, livestock, and domestic mammals. In one embodiment, the mammal may be a human, in others, the mammal may be a rodent, such as a mouse or a rat. In another embodiment, the subject is an animal modeled for cancer. Alternatively, the subject is a cancer patient.

The invention provides a method for diagnosing a tumor. The method comprises administrating an effective amount of gold particles to a subject in need thereof, and observing the distribution of the gold particles in the subject.

The gold particle coated with polymer has high selective accumulation and long retention in cancerous regions. Thus, the gold particles of the invention can reach much higher concentration in cancerous area to improve cancer therapy. The concentration of the gold particles in the tumor is higher than a concentration of the gold particle in a healthy tissue, such as 5 times higher than that in the healthy tissue. The gold particles concentrated in tumor areas can enhance the cell killing effects of radiotherapy, and the gold particles are easier excreted from healthy tissues, so that secondary damage is limited. In one embodiment, the concentration of the gold particle in the tumor is 21.2 times higher than the concentration of the gold particle in the healthy tissue at 36 hours after 170 mg/kg of the particle is intravenously rejected to the subject. In another embodiment, the concentration of the gold particle in the tumor is 30 times higher than the concentration of the gold particle in the healthy tissue at 12 hours after 170 mg/kg of the gold particle is intravenously rejected to the subject.

Since the gold particles of the invention have high biocompatibility, large quantity, and size homogeneity, it has a high selective accumulation in cancer regions and long retention there and in blood by the EPR effect.

The term “radiotherapy” refers to any therapeutic application of ionic radiation. The radiation may be radioactive radiation including, X-ray, ion beams, Gamma ray, fast electrons, neutrons, protons, Pi-mesons, microwaves, IR, or UV radiation. Preferably, the radiation is used to treat cancer or malignant hematopoietic diseases.

Further, because the gold particle of the invention is significantly aggregated in the tumor region, it also can be used as contrast enhancers for radiology in the diagnostic phase and during the assessment of the effects of therapy.

The gold particle of the invention can be orally, intravenously, or topically administrated to the subject; preferably, intravenously injection.

### EXAMPLE

### Example 1

### Synthesis of PEG-Gold Particles

PEGylated gold particles were synthesized from aqueous hydrogen tetrachloroaurate trihydrate (HAuCl₄.3H₂O, Aldrich) solutions by synchrotron X-ray irradiation. To achieve the best irradiation conditions, the pH value of the HAuCl₄.3H₂O solution was adjusted adding a NaOH solution. The synthesis of the gold particle was performed at the BL01A beam line of the NSRRC (National Synchrotron Radiation Research Center) storage rings for 5 minutes. The photon energy distribution was centered between 10 and 15 keV and the dose rate was 5.1±0.9 kGy/sec as determined by a Fricke dosimeter with an estimated G value of 13. To obtain well-dispersed PEGylated gold colloidal solutions, a mixed water solution of gold precursors (2 mM HAuCl4.3H2O, Aldrich, Mo., US) with appropriate NaOH (0.1 M, Showa Inc., Japan) and 3.3×10⁻⁵M polyethylene glycol (PEG) (MW 6000, Showa Inc., Japan) were placed into polypropylene conical tubes (15 ml, Falcon®, Becton Dickinson, N.J.) and transferred to the facility for X-ray irradiation.

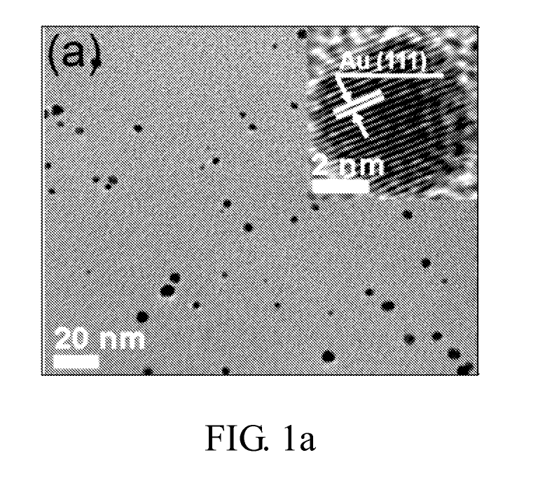

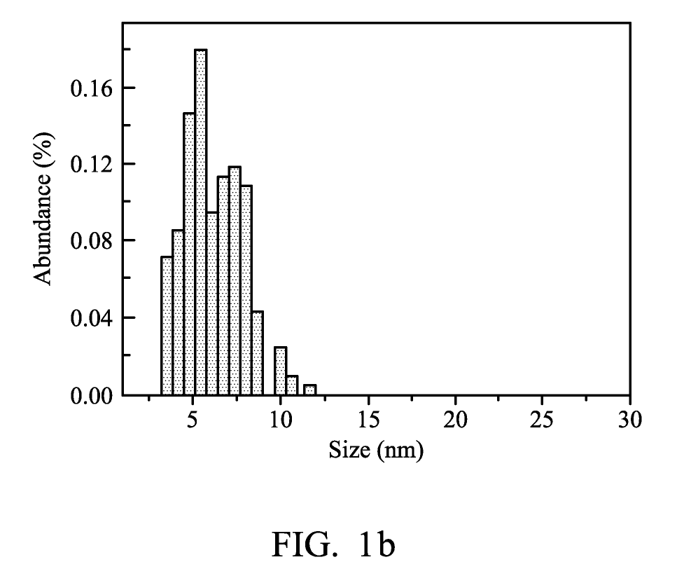

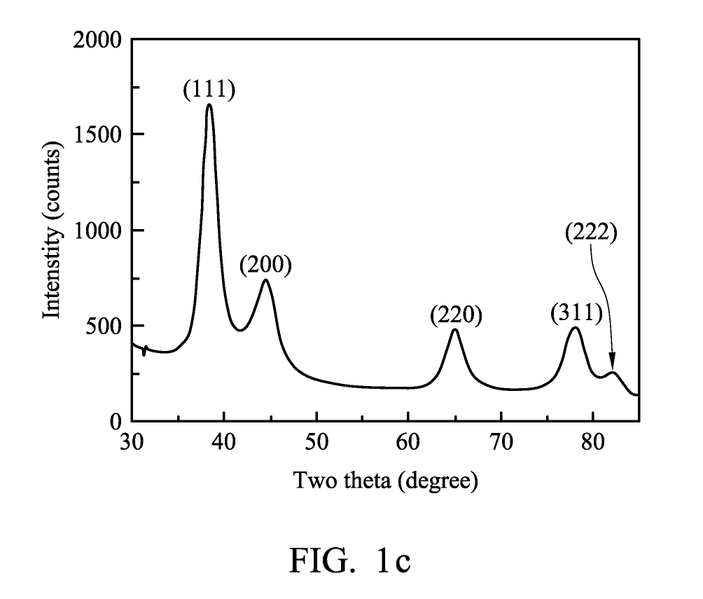

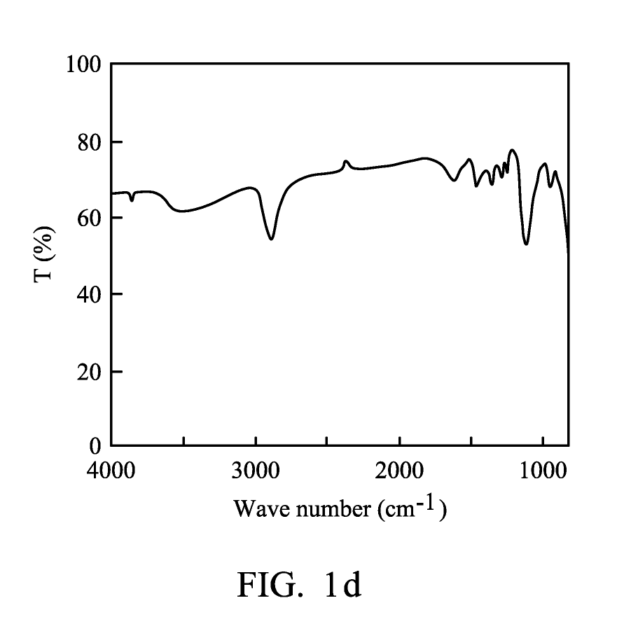

FIG. 1a illustrates TEM micrographs of the gold particles prepared by Example 1. The inset high-resolution image in FIG. 1a shows the Au (111) plane with a planar spacing of 2.32 Å confirming the crystal structure of the nanoparticles (note that High-resolution transmission electron microscopy (HRTEM) cannot reveal the adsorbed PEG because of its low electron density). Referring to FIG. 1b, after the solution was dried, the PEG-gold particles were spherical with 6.1±1.9 nm in diameter and reasonable size distribution. The crystal nature of PEG-gold particles was also confirmed by X-ray diffraction (XRD) measurements as shown in FIG. 1c. Referring to FIG. 1c, the estimated average particle size from the broadening of the reflection peak (111) was about 7.6 nm. The hydrodynamic size measured by dynamic light scattering for re-dispersed PEG-gold in de-ionized water was 27.9±8.1 nm. The thickness of the PEG coating was about 25 nm. A Fourier transform infrared (FTIR) spectrum of PEG-gold is shown in FIG. 1d. Referring to FIG. 1d, the broad band #1 in the 3200-3600 cm⁻¹ region was attributed to the OH group and suggests that hydroxyl plays a role in linking the gold particles to the PEG chains. Since vibrational bands including the CH₂ group (band #2 at 2884 cm⁻¹) and the C—O—C stretching (band #3 at 1114 cm⁻¹) were also detected, it confirms that PEG chains were immobilized at the particle surfaces.

### Example 2

### Observation of Cell Uptake

Mice colorectal adenocarcinoma CT26 cells (CRL-2638, ATCC, Rockville, Md.) were cultured in RPMI-1640 (Gibco, Invitrogen Corp., Carlsbad, Calif.) medium containing 10% fetal bovine serum (FBS) at 37° C. in a humidified 5% CO₂ incubator. The cells grew to 80% confluence and were detached by trypsin (0.5 g porcine trypsin and 0.2 g EDTA.4Na per liter of Hanks' Balanced Salt Solution) (Sigma, Saint Louis, Mo.). For the TEM sample preparation, 1×10⁵ CT-26 cells were seeded on a 100 mm culture dish. After 24 hours, an appropriate volume of concentrated PEGylated gold particles was added to the culture media to achieve a final colloidal concentration of 500 μm. After co-incubation of 48 hours, the cells with gold particles were trypsinized, centrifuged, and washed with PBS/5% sucrose for at least three times to remove the remaining particles. Subsequently, the cells were fixed for 2 hours in 2.5% glutaraldehyde, and postfixed for 2 hours in 1% osmium tetroxide. Dehydration was achieved by a 25%, 50%, 75%, 95%, and 100% ethanol solution. The samples were then infiltrated and embedded in 100% resin. Ultrathin sections prepared by an ultramicrotome were placed on 200-mesh copper grids for TEM measurement. Cells grown on glass slides and fixed with 2% paraformaldehyde for 15 minutes were imaged by confocal microscopy (with an Olympus FV-1000 system), wherein the cell nuclei, stained with a fluorescent dye (Hoechst 33258), were clearly observed.

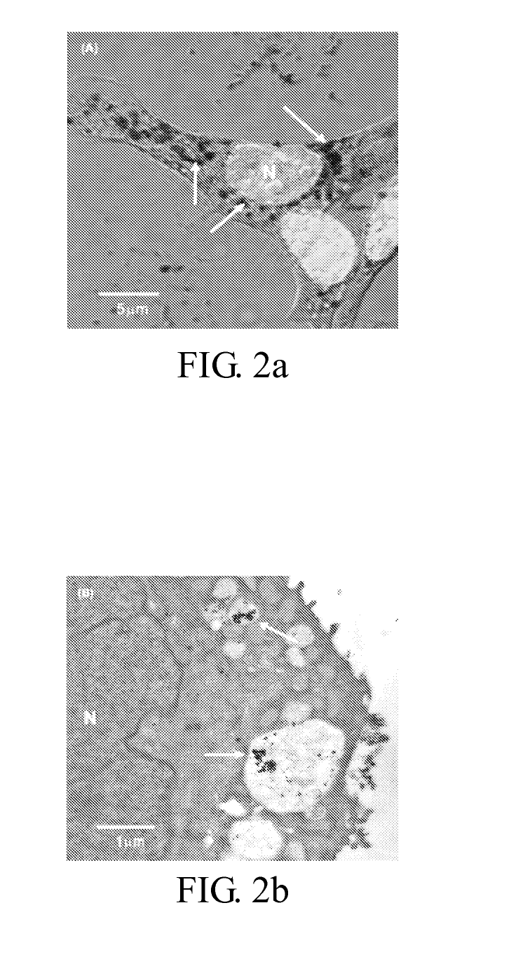

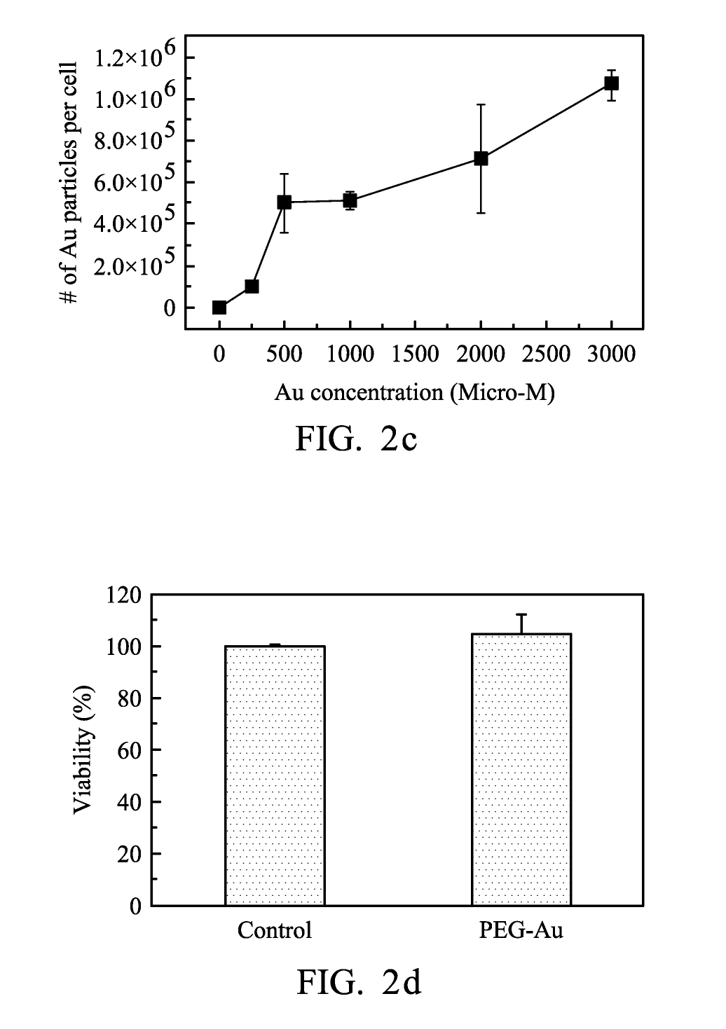

Referring to FIGS. 2a-2b, large amounts of Au particles were internalized in the cytoplasm. Specifically, all particles were clearly seen inside vesicles within the cytoplasm, no particles were detected inside the cell nucleus, and most particles were agglomerated.

For quantitative analysis of the cellular uptake of PEGylated gold particles, CT26 cells were seeded in 6-well microplates at a density of 2×10⁴ cell/well. After 24 hours of cell attachment, the cells were treated with different concentrations of colloidal PEGylated gold, and a quantitative analysis of gold particles was performed by ICP assay. A colonogenic cell survival test was performed with a radio-oncology linear accelerator (Clinac IX, Varian Associates, Inc., PaloAlto, Calif.) operating at 6 MV and with a dose rate of 2.4 Gy/min. 150 CT-26 cells/well were seeded and grown in a 6-well culture dish for 24 hours. PEGylated gold particles in the colloid solution (500 μM) were then introduced and retained for 48 hours, followed by irradiation by a 2 Gy dose of X-rays. The irradiated cells were further incubated for 14 days. Finally the cells were stained by 0.4% crystal violet and colonies were counted.

FIG. 2c shows that after co-culture for 48 hours, the amount of cellular uptake is dependant on the concentration of the added gold, wherein the uptake was about 0.5×10⁵ particles per cell at 500 μM and increased to more than 1.0×10⁶ particles per cell at 3000 μM. Further, standard cell viability tests demonstrated that the particles were not toxic, as shown in FIG. 2d.

### Example 3

### Viability Assay in Vivo

For RS 2000 biological irradiator and linear accelerator irradiation, 100 EMT-6 cells/well were seeded and grown in a 6-well culture. For the laboratory-based Cu Kα₁ X-ray and monochromatic synchrotron X-ray irradiation, 100 EMT-6 cells/well and CT-26 cells/well were seeded and grown in a 24-well culture and in a 48-well culture. 24 hours after cell seeding, PEG-gold particles (400 or 500 μM) were introduced and kept for 48 hours before X-ray irradiation. After irradiation, the cells were further incubated for 14 days. Finally the cells were stained by 0.4% crystal violet and colonies were counted.

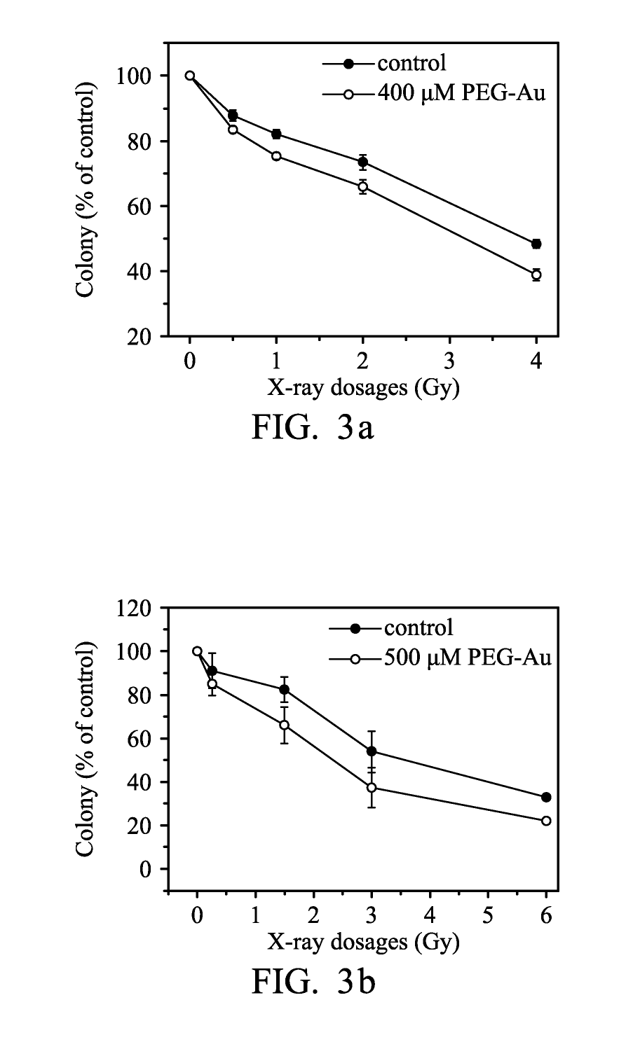

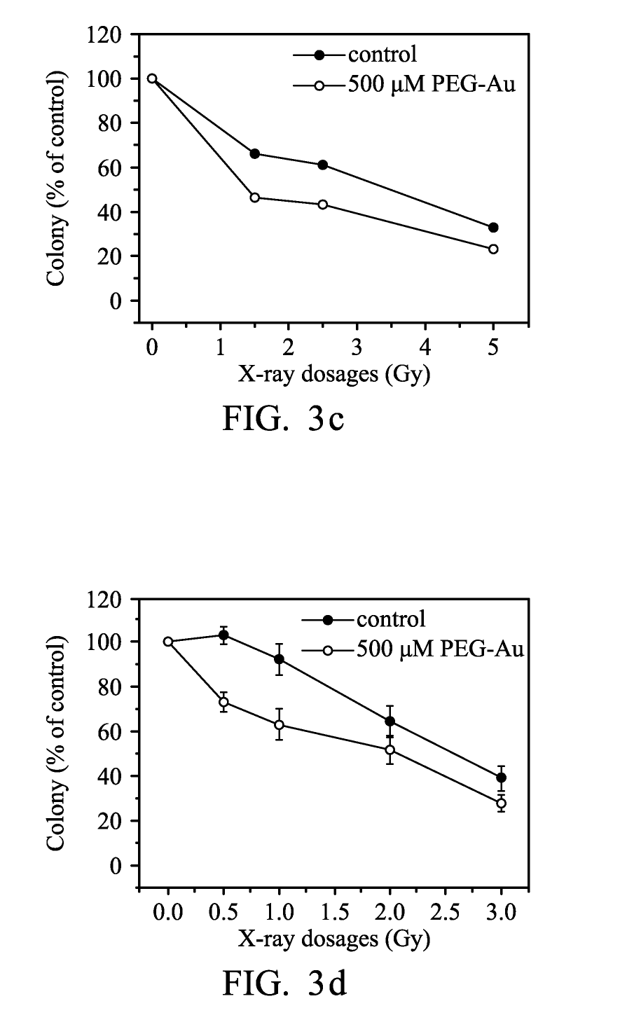

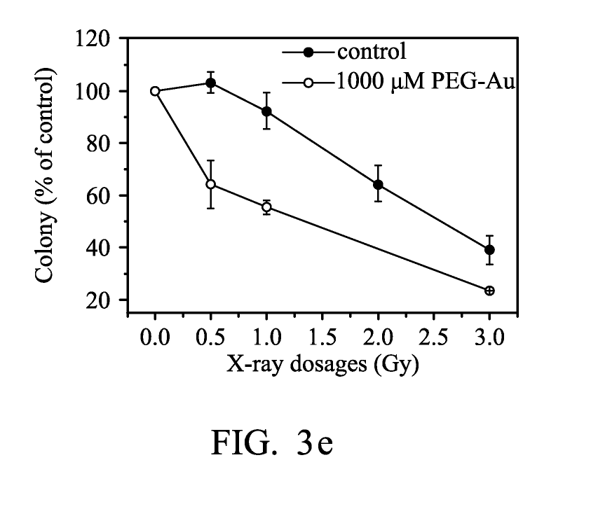

FIG. 3a is a graph plotting X-ray dosage against colony number (EMT-6 cell), wherein the full dots refer to control cells without PEG-gold particles, and the open circles refer to cells cultured in the presence of 400 μM of PEG-gold particles. Referring to FIG. 3a, after a irradiation of 1 or 2 Gy, the survival rate of EMT-6 cells was 84% and 76% for the control cells and decreased to 75% and 68% for cells exposed to PEG-Au particles. A similar pattern was found for all remaining doses.

FIGS. 3b-3c are graphs plotting X-ray dosage against colony number (EMT-6 cell), wherein X-ray includes Cu Kα₁ X-ray (FIG. 3b) and monochromatic synchrotron X-ray (FIG. 3c), respectively. Referring to FIGS. 3b-3c, the radiation decreased the survival rate of EMT-6 cells by 5.6% to 20.2%.

FIGS. 3d-3e are graphs plotting X-ray dosage against colony number (EMT-6 cell), wherein the concentration of the PEG-gold particle is 500 μm (FIG. 3d) and 1000 μm (FIG. 3e), respectively. Referring to FIGS. 3d-3e, the radiation decreased the survival rate of EMT-6 cells by 11.9% to 39%.

### Example 4

### Distribution of PEG Gold Particles in vivo

EMT-6 syngeneic mammary carcinoma cell lines were cultured under standard conditions. Male BALB/c mice (20-25 g, 6-8-week-old) were obtained from the National Laboratory Animal Center (Taiwan). The BALB/c ByJNarl tumor models were generated by inoculating 1×10⁶ EMT-6 cells in 10 μl PBS into the thigh of mice. The mice were used for the study 1 week after inoculation, when the tumor had grown to 50-90 mm³ (estimated as half the product of the square of the smaller diameter multiplied by the larger diameter). All animal experiments were performed according to the guidelines approved by the Laboratory Animal Care and Use Committee of Academia Sinica (Taiwan). Three individual bio-distribution experiments were performed with different injected doses of PEG-gold: 170, 231, and 488 mg/kg. The tumor-bearing mice were sacrificed at a given time points at 5 min, 10 min, 30 min, 90 min, 4 hr, 12 hr, 24 hr, and 36 hr after the colloidal injection. After sacrifice, important organs or tissues (blood, lung, tumor, muscle, brain, heart, liver, spleen and kidney) were collected for gold analysis by ICP-OES (Inductive Coupled Plasma-Optical Emission Spectroscopy).

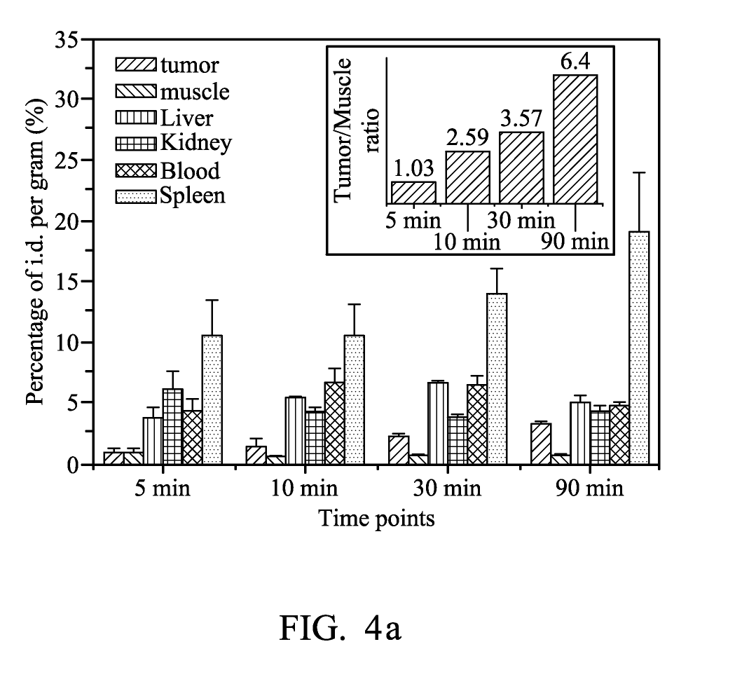

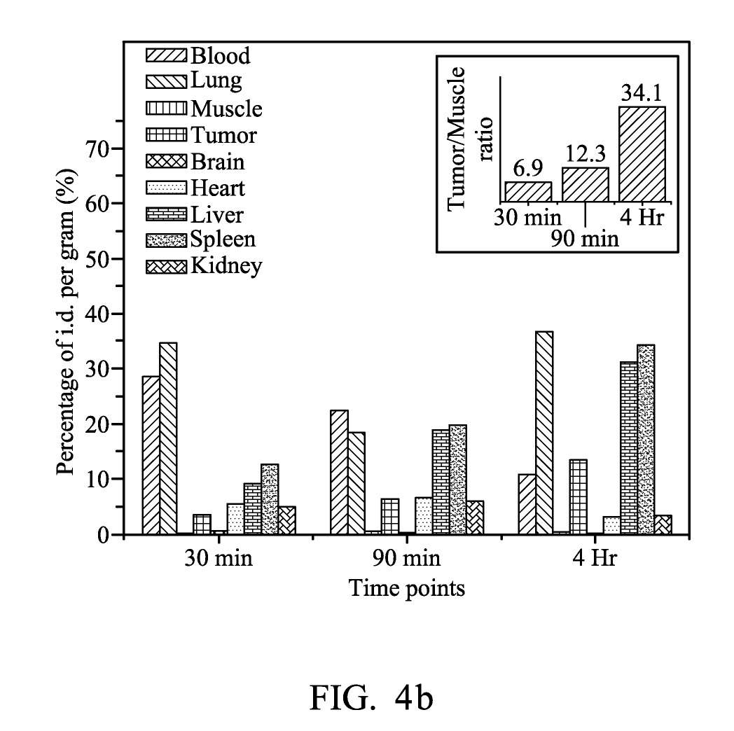

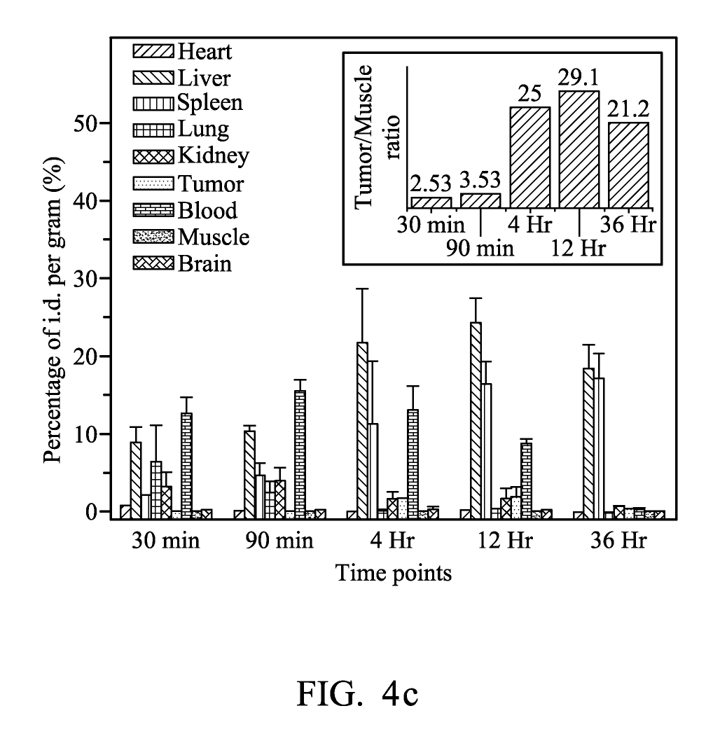

FIG. 4a shows the time-dependent distribution profiles of 6 nm PEG-gold colloidal at the largest administrated dose (488 mg/kg). At 5 min, there were only trace amounts of gold particles in either tumors or muscles. At 90 min, the gold concentration in all tumors monotonically was increased. On the contrary, gold particle amounts in muscles remain unchanged or decayed so that the tumor/muscle ratio showed a linearly increasing pattern reaching 6.4 at 90 min.

FIG. 4b shows the time-dependent distribution profiles of 6 nm PEG-gold colloidal at a middle administrated dose (231 mg/kg) for 30 minutes to 4 hours. Referring to FIG. 4b, at 4 hours, the tumor/muscle ratio reached 34.1. As described in FIG. 4c, for the lowest administrated dose (170 mg/kg), gold particle accumulation in tumor reached a maximum at about 12 hr after injection and gradually decayed following a longer time period. The gold particle concentration in the blood was found to decrease with time, which is consistent with other pharmacokinetic studies [J. F. Hainfeld, D. N. Slatkin and H. M. Smilowitz, Phys. Med. Biol. 49 (2004) N309; J. F. Hainfeld, D. N. Slatkin, T. M. Focella and H. M. Smilowitz, Br. J. Radiol. 79 (2006) 248].

The pharmacokinetics of uptake of PEG-gold nanoparticles by a RES system was also affected by the injected dose. The blood half time clearance at three administrated doses were estimated as >1.5 hr, about 4 hr and >12 hr for 488 mg/kg, 231 mg/kg and 170 mg/kg, respectively.

### Example 5

### Real-Time Analysis of the EPR Effect by Microradiology in vivo

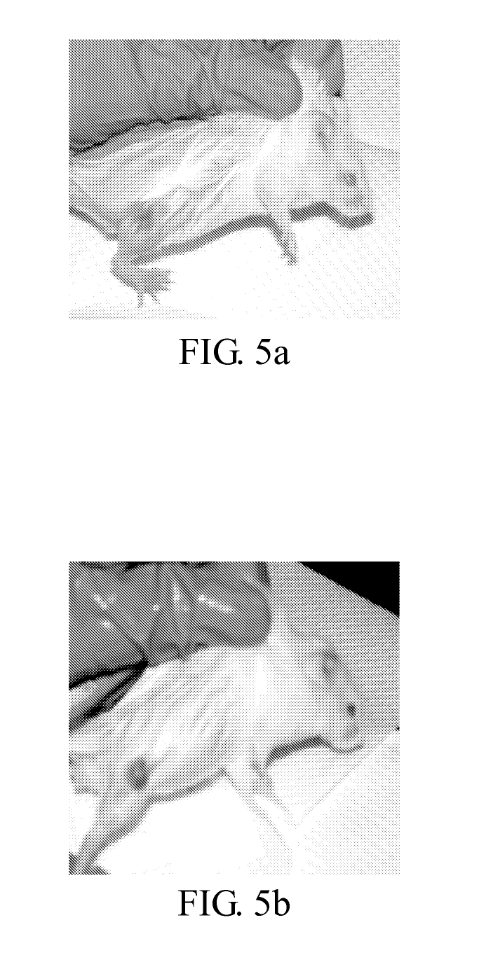

FIG. 5 shows a visual examination of the tumors. Referring to FIG. 5, a strong accumulation of gold particles in tumor was observed.

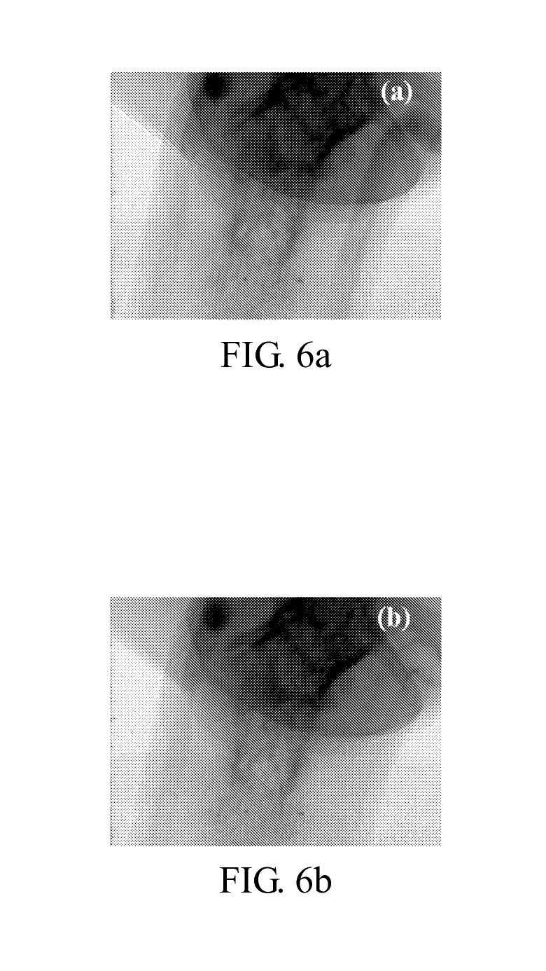

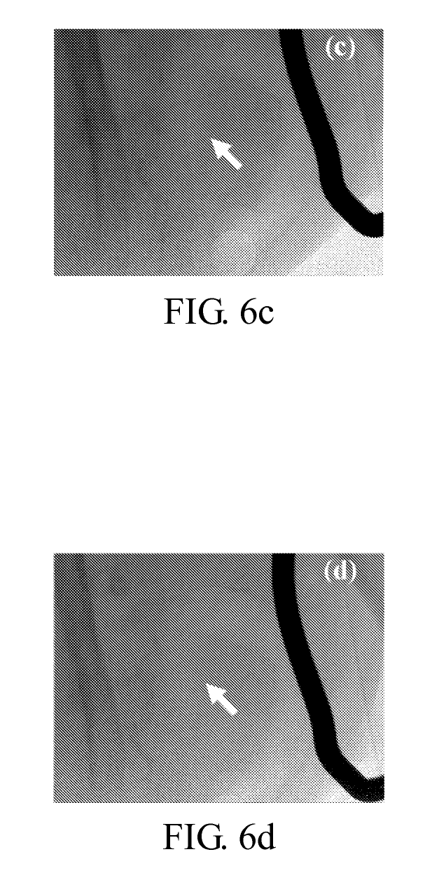

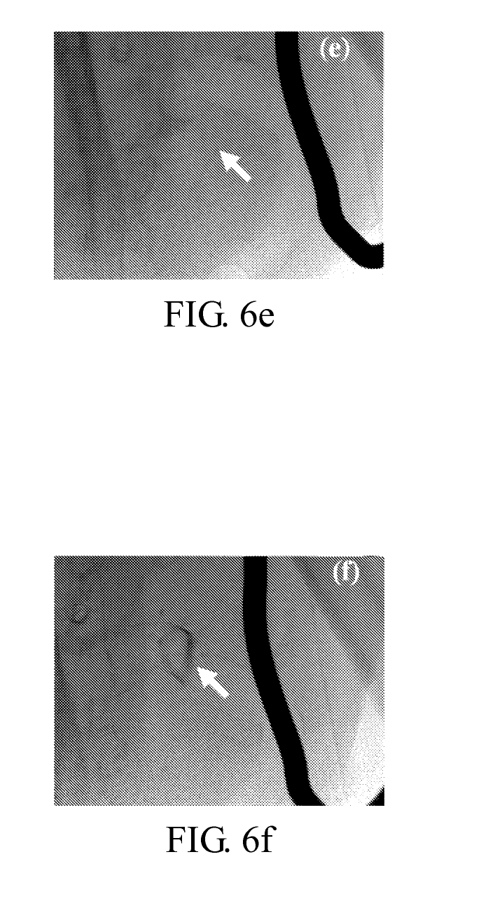

FIG. 6 shows a time sequence of microradiographs extracted from real-time video sequence taken during and after the injection of PEG-gold particles. Real time microradiology observations were performed at the BL01A beam line of the NSRRC (National Synchrotron Radiation Research Center) storage rings. The time of the each frame was 3 ms and the field of view (FOV) was 3 mm. The images were captured during and after the injection of 100 μl (˜75 mg/ml) of gold nanosols via the tail vein of the mice by a syringe pump. Before the test, the mice were gently anaesthetized. FIGS. 6a and 6b show the tail region of the mice at 3 sec (FIG. 6a) or 4 sec (FIG. 6b) after injection, showing that the injected particles (dark) passed through the blood vessels. FIGS. 6c to 6f show a region containing tumor and blood vessels at 10 sec (FIG. 6c), 2 min (FIG. 6d), 10 min (FIG. 6e), and 15 min (FIG. 6f) after injection, respectively. Approximately 10 sec after the injection, the configurations of the main vein vessel, microvasculature and tumor sites was clearly revealed by the darkening effect of the accumulated particles. After 2 min, both the tumor outlines and the intra-tumor tissue structure were observed with increasing contrast.

The strongest contrast appeared 15 min after injection. The tumors became clearly visible with no image processing. However, compared to the 50 sec images of the tail region, the boundaries of the large vessels were less visible. This indicates that between 50 and 100 sec the gold accumulation in the large vessels was depleted whereas the accumulation in the tumor and in the nearby microvascularization increased. This indicates that the EPR effect for different organs has different and sometimes complex time evolutions, not revealed by mere visual inspection.

### Example 6

### Microscopic-Scale TEM Analysis of the Gold Particles Distribution

To reveal the PEGylated gold particle distribution, TEM samples were prepared. Firstly, after the scarification of the mice, the organs were immediately fixed with glutaraldyhyde at 4 C for 24 hrs. After replacing the glutaraldyhyde by 0.1 M PBS, the samples were further fixed and stained with 1% osmium tetraoxide in a buffer and dehydrated by a series of alcohol treatments, embedded in resin, and sliced to 90-100 nm in thicknesses using a Leica Ultracut R ultramicrotone. After being double stained with uranyl acetate and lead citrate, the specimens were observed by a Hitachi H-7500 TEM operating at 100 keV.

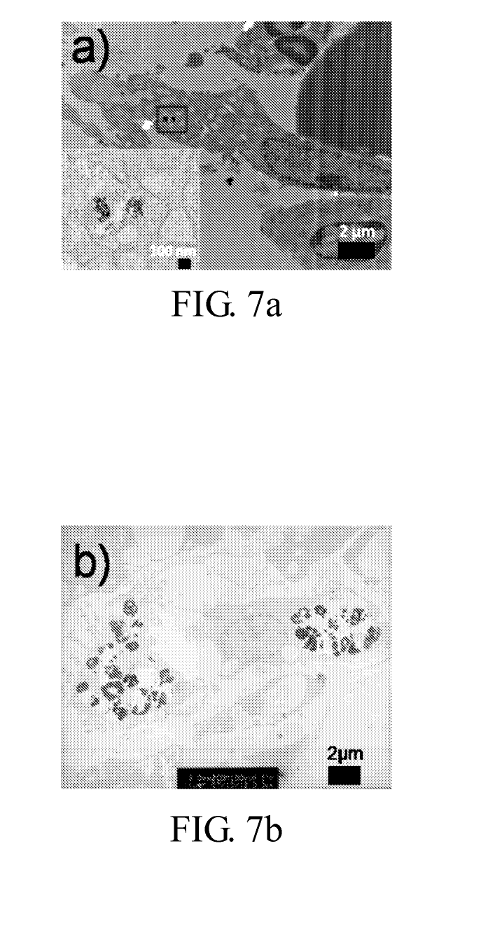

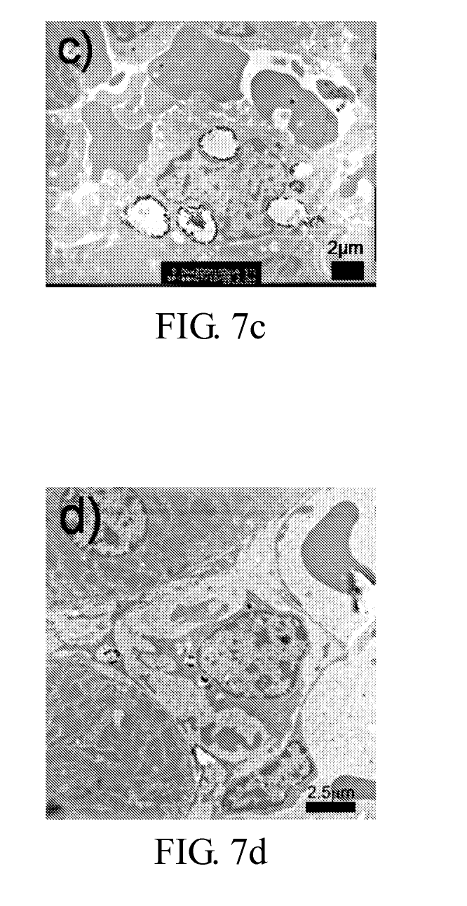

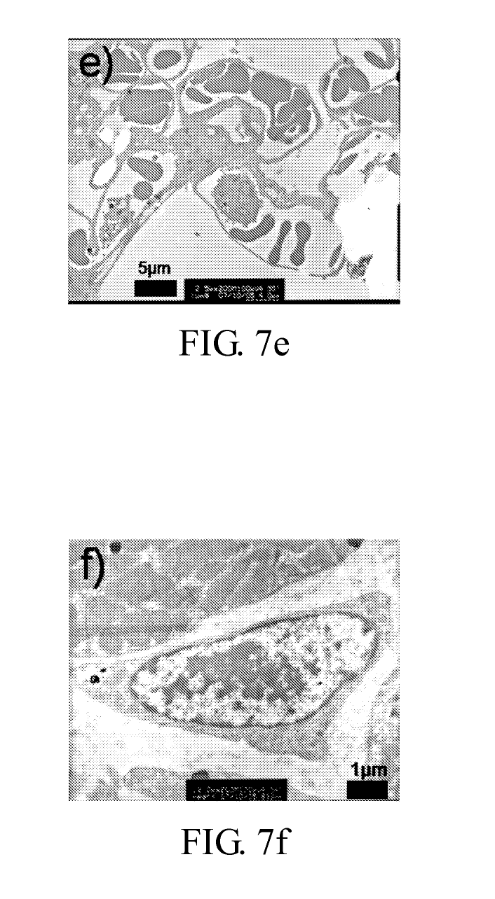

TEM micrographs of various mice organs and tumor are shown in FIG. 7, wherein FIG. 7a is an image of a tumor region, FIG. 7b is an image of a liver region, FIG. 7c is an image of a spleen region, FIG. 7d is and image of a kidney region, FIG. 7e is an image of a lung region, and FIG. 7f is an image of a heart region. The individual gold particle size was 30-40 nm, much larger than the originally administrated 6 nm. By analogy with other experiments, the increase of the size related to colloidal flocculation and eventual aggregation with other small gold particles. It was found that gold particles aggregated in the endosome were almost confined within the cytoplasm of the liver cells, and the individual particles size was 50-100 nm.

### Example 7

### Histology Imaging Examination in vivo

To examine the pathological characteristics of PEG-gold loaded organs/tissues, different organs—tumor, spleen, liver, lung, kidney and muscle—were immediately fixed in 10% formalin and dehydrated by a series of immersions in a 50%, 70%, 90% and 100% ethanol solution. They were then embedded in paraffin wax and sectioned to 2-5 μm slices with a Leica RM2235 microtome. After histological H-E staining, the slices were observed by a confocal laser scanning microscope (Leica TCS-ST, Germany).

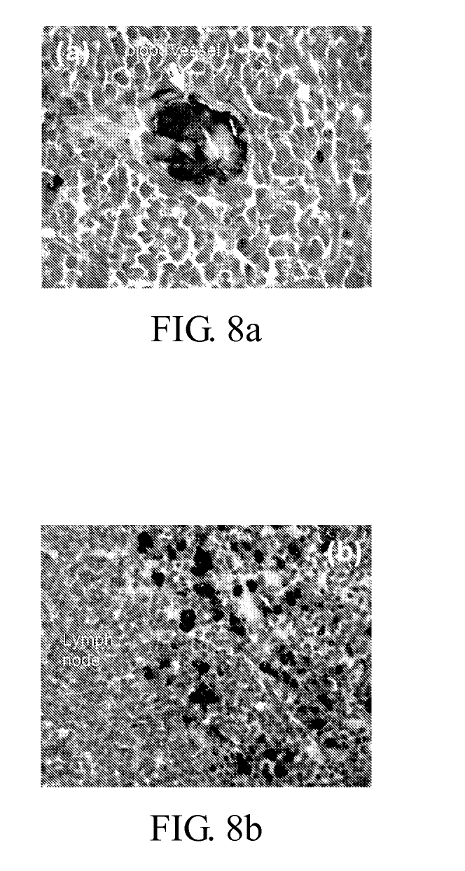

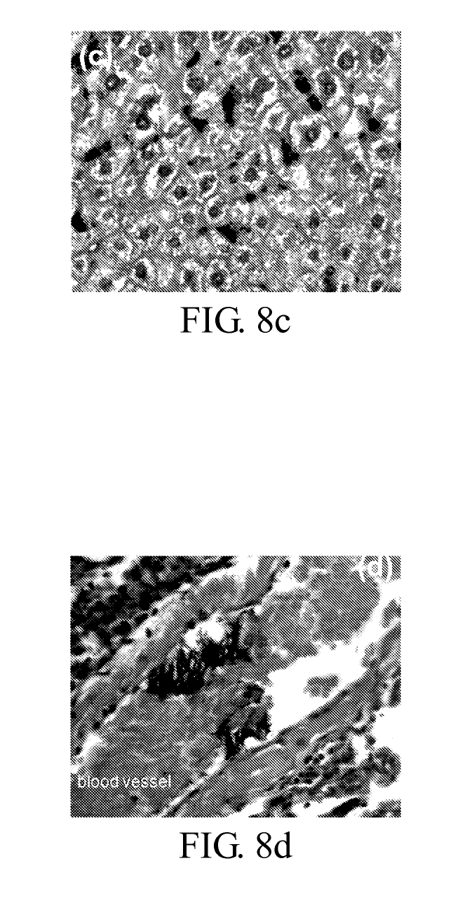

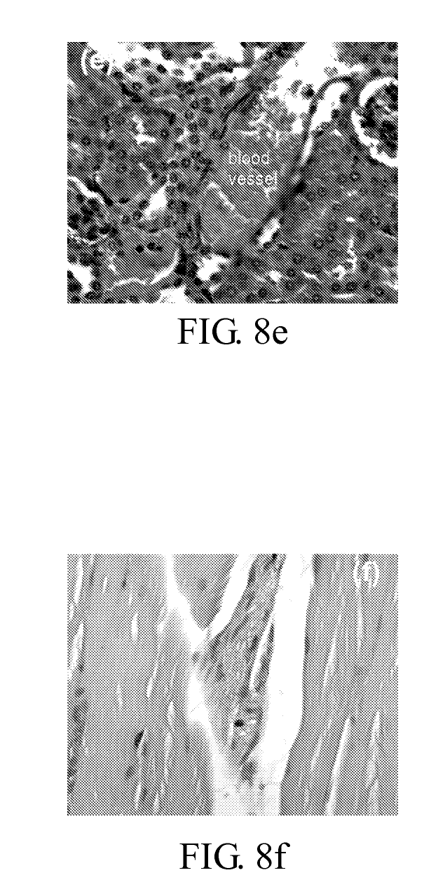

FIG. 8 shows microscopy images of tumor, spleen, liver and lung, kidney, and muscle after H-E staining. FIG. 8a shows a cross-section of a small blood vessel in a tumor containing region. The vessel is filled with gold particles (dark regions). Some particles were also observed in the neighboring inter-cellular matrix.

Referring to FIG. 8b, the accumulated particles were mostly observed within the red pulp in spleen.

FIG. 8c shows the gold particle distribution within the network-like lobules of liver. The image indicates the formation of particle aggregates. The hypatocytes within the lobule captured the particle aggregates that accumulated within both the eosinophilic cytoplasm and the interface region.

Referring to FIG. 8d, flake-like large gold aggregates also appeared within the non-tumor micro-vasculature of the lung. The aggregates were mostly close to vessel walls.

Referring to FIG. 8e, the kidney had less gold particles than the lungs, and the gold particles accumulated within the inner microvasculature and the renal cortex.

Referring to FIG. 8f, some particles were present in the muscle tissue, but not in the skeletal muscle fibers, eosinophilic cytoplasm, and the small peripheral nuclei.

### Example 9

### Enhancement Effect on the Suppression of the Tumor Growth in Mice

8 Balb/C mice (20 g) were injected subcutaneously in the thigh with 5×10⁶ EMT-6 syngeneic mammary carcinoma cells performed on a RS 2000 x-ray Biological Irradiator (RadSource Tech. Inc., Boca Raton, Fla.) working at 160 kV and 25 mA with an average photon energy of about 73 KeV. The mean dose rate was 0.037 Gy/sec. After 1 week, 3 mg (0.2 cc Au, 25 mg/ml) of PEG-Au particles was introduced via tail vain injection. 12 hours after PEG-Au particles injection, a radiation dose of 10 Gy was applied for the tumor treatment. The tumor volume was t monitored every 3 days till to 3 weeks by the two perpendicular diameters with a vernier caliper. The tumor volume was calculated according to the below formula: The tumor volume=0.4(ab²). “a” is the length of the longer diameter and “b” is the length of shorter diameter.

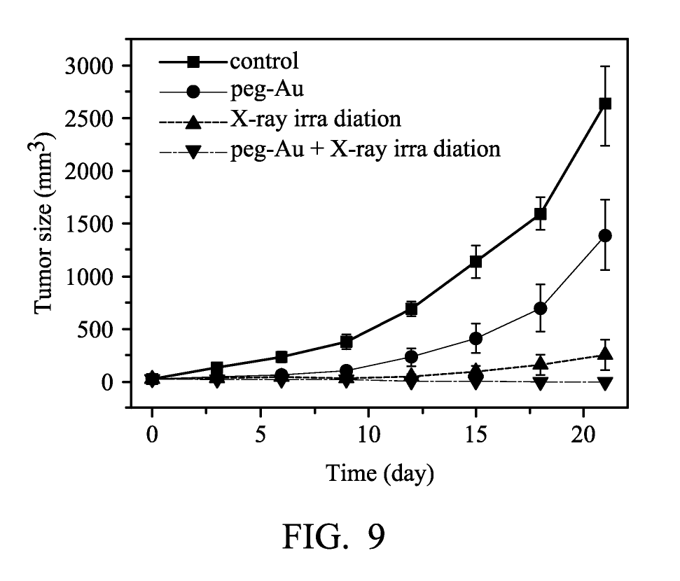

The results clearly reveal the particle enhancement effect on the suppression of the tumor growth by x-ray irradiation as shown in FIG. 9. For example, at the time point of 21 day after X-ray treatment, the tumor volume was about 250 mm³ for the control mice. While for the PEG-Au+X-ray irradiation system, the mice had no visible tumor, indicating that the injected tumor mice is cured under this condition.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for diagnosing a tumor, comprising
administrating an effective amount of gold particles to a subject in need thereof, and
observing the distribution of the gold particles in the subject, wherein the gold particles are coated with a polymer, and the gold particle has a size of about 6.1±1.9 nm.

2. The method as claimed in claim 1, wherein the polymer comprises PEG, PEI, PVP, or IPA.

3. The method as claimed in claim 1, wherein the gold particles are thiol-free gold particles.

4. The method as claimed in claim 1, wherein the gold particles are thiol-free PEGylated gold particles.

5. The method as claimed in claim 1, wherein a concentration of the gold particles in the tumor is higher than a concentration of the gold particles in a healthy tissue.

6. The method as claimed in claim 5, wherein the concentration of the gold particle in the tumor is 21.2 times higher than the concentration of the gold particle in the healthy tissue at 36 hours after 170 mg/kg of the particle is intravenously rejected to the subject.

7. The method as claimed in claim 5, wherein the concentration of the gold particle in the tumor is 30 times higher than the concentration of the gold particle in the healthy tissue at 12 hours after 170 mg/kg of the particle is intravenously rejected to the subject.

8. The method as claimed in claim 1, further irradiating the tumor with radiation for treating the tumor.

9. The method as claimed in claim 8, wherein the radiation comprises X-ray, ion beams, Gamma ray, fast electrons, neutrons, protons, Pi-mesons, microwaves, IR, or UV radiation.

10. The method as claimed in claim 1, wherein the subject is a mammal.

11. The method as claimed in claim 1, wherein the gold particles are orally, intravenously, or topically administered.

12. The method as claimed in claim 1, wherein the tumor comprises a breast cancer, a lung cancer, a brain cancer, a liver cancer, a skin cancer, a kidney cancer, a GI cancer, a prostate cancer, a bladder cancer, or a gynecologic cancers.
